(19) United States
(12) Patent Application Publication
(10) Pub. No.: US 2018/0143455 A1
(43) Pub. Date: May 24, 2018

(54) SWITCHABLE LENS DEVICES, SYSTEMS, AND RELATED METHODS

(71) Applicant: ELWHA LLC, BELLEVUE, WA (US)

(72) Inventors: Hon Wah Chin, Palo Alto, CA (US); Roderick A. Hyde, Redmond, WA (US); Katherine E. Sharadin, Redmond, WA (US); Clarence T. Tegreene, Mercer Island, WA (US)

(21) Appl. No.: 15/358,843

(22) Filed: Nov. 22, 2016

(51) Int. Cl. G02C 7/08; G02C 7/04; G02C 7/02

(52) U.S. Cl. CPC G02C 7/083; A61F 2/16; G02C 7/02; G02C 7/04

(57) ABSTRACT

Embodiments disclosed herein are directed to switchable lens devices, systems, and methods related to detecting one or more commands and switching one or more switchable lenses responsive to one or more detected commands.

### RELATED METHODS

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### BACKGROUND

Intraocular lenses (IOLs), such as pseudophakic IOLs, aphikic IOLs, or phakic IOLs (PIOLS), can be used to correct the vision of a subject. Typical IOLs can include monofocal, multifocal, or accommodative configurations. IOLs can include an optic element (e.g., lens) and haptic elements (e.g., arms or wings configured to aid in positioning the IOL).

Such configurations can be limited to focusing either on near or far vision without selectively modifiable adjustment therebetween. Therefore, manufacturers, users, and designers of IOLs continue to seek improved IOLs.

### SUMMARY

Embodiments disclosed herein are directed to switchable lens devices and systems, such as IOL devices and IOL systems, and methods that include controlling the switching of the switchable lenses. For example, as described below in more detail, the switchable lenses can have multiple optical settings and can be switched from one optical setting to another (e.g., responsive to one or more switching signals from a controller). In an embodiment, the controller can receive one or more inputs from a user and can switch or direct switching of one or more switchable lenses. For example, the controller can automatically (e.g., without a command from the user or responsive to a signal from one or more sensors detecting a non-command input, event, or condition) switch the switchable lens from a first optical setting to a second optical setting, and can switch back to the first optical setting, from the second optical setting, responsive to an override command or input received from the user (e.g., from the subject wearing the switchable lens).

An embodiment includes a lens system that includes at least one switchable lens device including at least one switchable lens configured to selectively switch between a first optical setting and at least a second optical setting, and one or more sensors configured to detect at least one focus-related characteristic of one or more of a first eye or a second eye of the subject or at least one focus-related characteristic of an environment proximate to the subject. The lens system also includes a controller operably coupled to the one or more sensors and operably coupled to the at least one switchable lens device. The controller includes control electrical circuitry that is configured to receive one or more sensor signals from the one or more sensors, and direct sensor-based switching of the at least one switchable lens from the first optical setting to the at least a second optical setting responsive to the one or more received sensor signals. Moreover, the control electrical circuitry of the controller is configured to receive one or more inputs from the subject and, after directing sensor-based switching of the at least one switchable lens from the first optical setting to the second optical setting, direct override switching of the at least one switchable lens from the at least a second optical setting to the first optical setting at least partially responsive to the one or more inputs received from the subject.

An embodiment includes a lens system that includes at least one switchable lens device including at least one switchable lens configured to selectively switch between a first optical setting and at least a second optical setting, and one or more sensors configured to detect at least one focus-related characteristic of one or more of the first eye or the second eye of the subject or of an environment proximate to the subject. The lens system also includes a controller operably coupled to the one or more sensors and operably coupled to the at least one switchable lens device. The controller includes control electrical circuitry that is configured to receive one or more sensor signals from the one or more sensors and direct sensor-based switching an optical setting of the at least one switchable lens from the first optical setting to the at least a second optical setting or from the second optical setting to the first optical setting responsive to the one or more sensor signals, when a state setting is set to an enabled state, and to maintain the optical setting unchanged, when the state setting is set to a disabled state. Moreover, the control electrical circuitry of the controller is configured to receive one or more inputs from the subject and, after sensor-based switching the optical setting of the at least one switchable lens, at least partially responsive to the one or more inputs received from the subject, change the state setting between a disabled state and an enabled state.

An embodiment includes a method of changing an optical setting of one or more switchable lens devices. The method includes, at a controller, receiving one or more sensor signals from one or more sensors. The one or more sensor signals are related to a change in vergence between a first eye and a second eye of the subject. The method also includes directing sensor-based switching of at least one switchable lens from a first optical setting to at least a second optical setting responsive to the one or more received sensor signals. Moreover, the method includes after sensor-based switching the at least one switchable lens from the first optical setting to the at least a second optical setting, receiving one or more inputs from the subject and, at a controller, directing override switching of the at least one switchable lens from the second optical setting to the first optical setting at least partially responsive to the one or more inputs received from the subject.

An embodiment includes a method of changing an optical setting of one or more switchable lens devices. The method includes, at a controller, receiving one or more sensor signals from one or more sensors. The one or more sensor signals are related to a change in vergence between a first eye and a second eye of the subject. The method also includes receiving one or more inputs from the subject and changing a state setting from a disabled state to an enabled state or from an enabled state to a disabled state at least partially responsive to the one or more inputs received from the subject. Moreover, the method includes, after changing the states from the disabled state to the enabled state responsive to the one or more user inputs, directing switching an optical setting of at least one switchable lens from a first optical setting to a second optical setting or from the second optical setting to the first optical setting responsive to the one or more sensor signals, when the state setting is set to the enabled state, and to maintaining the optical setting unchanged, when the state setting is set to the disabled state.

An embodiment includes a lens system that includes at least one switchable lens device including at least one switchable lens configured to selectively switch between a first optical setting and at least a second optical setting, and one or more sensors configured to detect at least one focus-related characteristic of one or more of a first eye or a second eye of the subject or at least one focus-related characteristic of an environment proximate to the subject. The lens system also includes a controller operably coupled to the one or more sensors and operably coupled to the at least one switchable lens device. The controller includes control electrical circuitry that is configured to receive one or more sensor signals from the one or more sensors, and direct sensor-based switching of the at least one switchable lens from the first optical setting to the at least a second optical setting responsive to the one or more received sensor signals and based on a first algorithm. The control electrical circuitry of the controller is configured to receive one or more inputs from the subject. The control electrical circuitry of the controller is configured to, after receiving the one or more inputs from the subject, stop directing sensor-based switching or direct sensor-based switching of the at least one switchable lens from the first optical setting to the at least a second optical setting responsive to the one or more received sensor signals and based on a second algorithm that is different from the first algorithm.

Features from any of the disclosed embodiments can be used in combination with one another, without limitation. In addition, other features and advantages of the present disclosure will become apparent to those of ordinary skill in the art through consideration of the following detailed description and the accompanying drawings.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic top view of a subject's eyes having a first vergence therebetween and focused on a first object at a first distance from the subject, according to an embodiment.

FIG. 2 is a schematic top view of the subject's eyes of FIG. 1, with the eyes having a second vergence therebetween and are focused on a second object at a second distance from the subject that is less than the first distance, according to an embodiment.

FIG. 3A is a schematic top view of a switchable lens device located in the eye, according to an embodiment.

FIG. 3B is a schematic side view of the switchable lens device of FIG. 3A.

FIG. 4 is a schematic front view of a switchable lens system worn by a subject, according to an embodiment.

FIG. 5 is a schematic top view of a switchable lens system, according to an embodiment.

FIG. 6A is a schematic front view of a switchable lens system worn by a subject, according to an embodiment.

FIG. 6B is a schematic top view of the switchable lens system of FIG. 6A.

FIG. 7 is a schematic front view of a switchable lens system worn by a subject, according to an embodiment.

FIG. 8 is a schematic side view of a switchable lens system worn by a subject, according to an embodiment.

FIG. 9 is a schematic side view of the switchable lens system, according to an embodiment.

FIG. 10 is a schematic diagram of a switchable lens system, according to an embodiment.

### DETAILED DESCRIPTION

Embodiments disclosed herein are directed to switchable lens devices and systems, such as IOL devices and IOL systems, and methods that include controlling the switching of the switchable lenses. For example, as described below in more detail, the switchable lenses can have multiple optical settings and can be switched from one optical setting to another (e.g., responsive to one or more switching signals from a controller). In an embodiment, the controller can receive one or more inputs from a user and can switch or direct switching of one or more switchable lenses. For example, the controller can automatically (e.g., without a command from the user or responsive to a signal from one or more sensors detecting a non-command input, event, or condition) switch the switchable lens from a first optical setting to a second optical setting, and can switch back to the first optical setting, from the second optical setting, responsive to an override command or input received from the user (e.g., from the subject wearing the switchable lens).

Generally, the automatic switching of the switchable lens or switching without a command from the subject can be performed responsive to one or more signals received at the controller and correlated to one or more indicators that a vergence between the subject's eyes has changed. As described below in more detail, the switchable lens device or system can include one or more sensors that can detect motion of subject's eyes or correlate motion of the subject's eye with change in vergence therebetween. For example, the controller can receive one or more signals from such sensors and can switch the switchable lens from a first optical setting (e.g., a first focal length) to a second optical setting (e.g., a second focal length).

In an embodiment, switching from one optical setting to another (e.g., from a first focal length to a second focal length) can include a predetermined or selected time delay therebetween. Specifically, for example, the controller can set a time for switching the switchable lenses between optical settings, as disclosed in more detail in U.S. Patent Application No. TBD, Attorney Docket No. 260577US01_499008-55, entitled “INTRAOCULAR LENS SYSTEMS AND RELATED METHODS, which is filed concurrently herewith, the entire content of which is incorporated herein by this reference. For example, a switching time can be received as an input (e.g., from the subject) or can be calculated based on one or more algorithms or formulas and can be stored by the controller. Moreover, the controller can apply the switching time to the switching of the switchable lenses responsive to sensor input or to the switching of the switchable lenses responsive to input received from the subject (e.g., responsive to commands, such as override commands, received from the subject).

The switchable lens system can include any number of suitable sensors that can provide one or more suitable signals to the controller, which can indicate to the controller a change in vergence between the subject's eyes. In an embodiment, the switchable lens systems disclosed herein can include one or more sensors configured to detect one or more physiological indicia of the subject. For example, the switchable lens system can include one or more sensors configured to detect glucose concentration (e.g., in the eye of the subject), eye pressure, heart rate, biological proteins present in the eye, or any other biological indicia. The one or more sensors can be operably coupled to the controller. The controller of the switchable lens system can be configured to transmit the measurements of the physical indicia to a remote source such as a computer, a cellular phone, or other electronic device. In an embodiment, the measured physical indicia may be used to determine the health of a subject or eye thereof, customize the operation of the switchable lens device to the particular subject, determine if the controller needs to be removed or adjusted, or determine if the focal adjustments of the controller are suitable for the subject. The electronic device may then transmit instructions to the controller to selectively control or otherwise adjust the functioning of the switchable lens system, such as controllably changing the focal length of the switchable lens device.

In some operating conditions, the subject may desire to have one or more of the switchable lenses switch to the previous optical setting (e.g., to the optical setting of the switchable lenses before the non-command switching described above). For example, the controller can include or can be operably coupled to one or more input devices or sensors that can detect or receive inputs from the subject (e.g., override inputs or commands, to change the current optical setting of the switchable lens to a previous optical setting). In an embodiment, the controller can be operably coupled to an audio detection device, such as a microphone (e.g., piezo microphone, laser microphone, condenser microphone, etc.). Hence, for example, the controller can receive one or more audible inputs and can correlate the audible inputs to one or more optical settings for the switchable lens(es) or to one or more switching commands. The controller can switch or direct switching of one or more switchable lenses responsive to the switching commands (e.g., switching command can be correlated to a specific optical setting for one or more of the switchable lenses).

In one or more embodiments, the controller can include or can be operably coupled to one or more motion or gesture detection sensors. For example, the sensors can detect one or more selected movements or gestures (e.g., gestures made by the subject). The controller can receive one or more signals from the motion or gesture detection sensors and can correlate subject's gestures or movements with one or more switching commands. For example, responsive to one or more switching commands received from the subject, the controller can change one or more optical settings of one or more of the switchable lenses, as described herein.

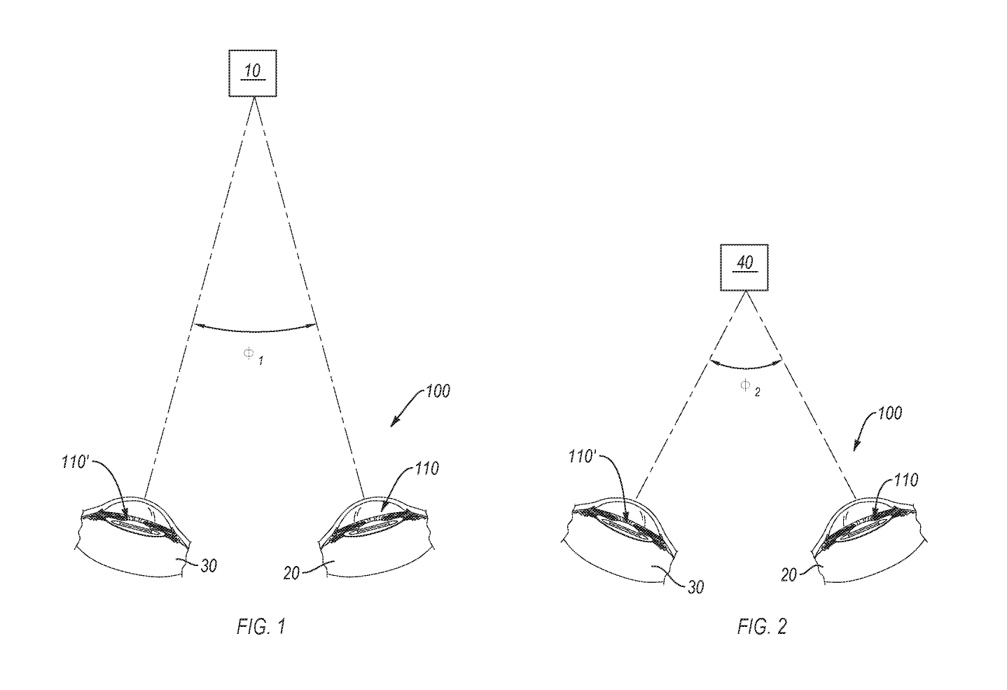

In an embodiment, the switchable lens can be configured to switch between two or more focal lengths. FIG. 1 schematically illustrates eyes 20 and 30 of a subject focused on a first object 10 that is positioned at a first distance from the subject. In particular, when the eyes 20, 30 are focused on the first object 10, an angle between respective optical axes thereof can be at a vergence angle ϕ₁. FIG. 1 also schematically illustrates a switchable lens system 100, according to an embodiment. For example, switchable lens system 100 can include a first switchable lens device 110 positioned in a first eye 20 and a second switchable lens device 110′ positioned in a second eye 30 of the subject.

Generally, the first switchable lens device 110 or the second switchable lens device 110′ can be configured to augment or correct visual deficiencies of the subject or to replace the lenses in the respective first eye 20 or second eye 30 of the subject (e.g., in cataract surgeries). It should be appreciated that, in one or more embodiments, the switchable lens system 100 can include only a single switchable lens device (e.g., the first switchable lens device 110 or the second switchable lens device 110′), which can be associated with or positioned in the first eye 20 or in the second eye 30. The switchable lens devices 110 or 110′ can be switched to or set at a first optical setting, such as a first focal length.

For example, at the first focal length, the light entering the eye from the distance of the first object 10 is focused on the retina of the respective eyes 20, 30, thereby focusing the eyes 20, 30 on the first object 10. Moreover, the switchable lens devices 110 or 110′ can be switched to a second optical setting, such as a second focal length. When the subject focuses on another object, such as an object that is closer to the subject than the first object 10, the subject's eyes 20, 30 can tilt such as to converge, thereby changing the angle between the optical axes thereof. FIG. 2 schematically shows the subject's eyes 20, 30 focused on a second object 40, which is positioned at a second distance and closer to the subject than the first object 10 (FIG. 1). For example, when the eyes 20, 30 focus on the second object 40, the angle between the optical axes thereof can change to a second angle ϕ₂. More specifically, as the eyes 20, 30 focus on the closer, second object 40, the eyes 20, 30 converge or in-tilt, such that the second angle ϕ₂ defined by the respective optical axis thereof is greater than the first angle ϕ₁.

In an embodiment, the switchable lens devices 110 or 110′ can be switched to the second focal length, which can be shorter than the first focal length. The switchable lens devices 110 or 110′ can include one or more sensors that can sense or detect a one or more instructions or inputs (e.g., one or more inputs from the subject) and can correlate the one or more inputs from the subject to a change in vergence rotation between the eyes 20, 30 (e.g., convergence to focus on a closer object or divergence to focus on a farther object) or to a request or command to change one or more optical settings of the switchable lens devices 110 or 110′ (e.g., to change a focal length, to change visible-light transmittance, to change focal spot, etc.).

Generally, switchable lenses can have any number of optical settings, and the controller can directly or indirectly select at least one of the optical settings for the switchable lens. Hence, for example, the switchable lens devices (e.g., the switchable lens device 110 or switchable lens device 110′) can be configured for switching among any suitable number of optical settings, as described below in more detail. In one or more embodiments, the switchable lens devices described herein may have optical settings with a single optical parameter (e.g., focal length, light transmittance, etc.) or multiple optical parameters.

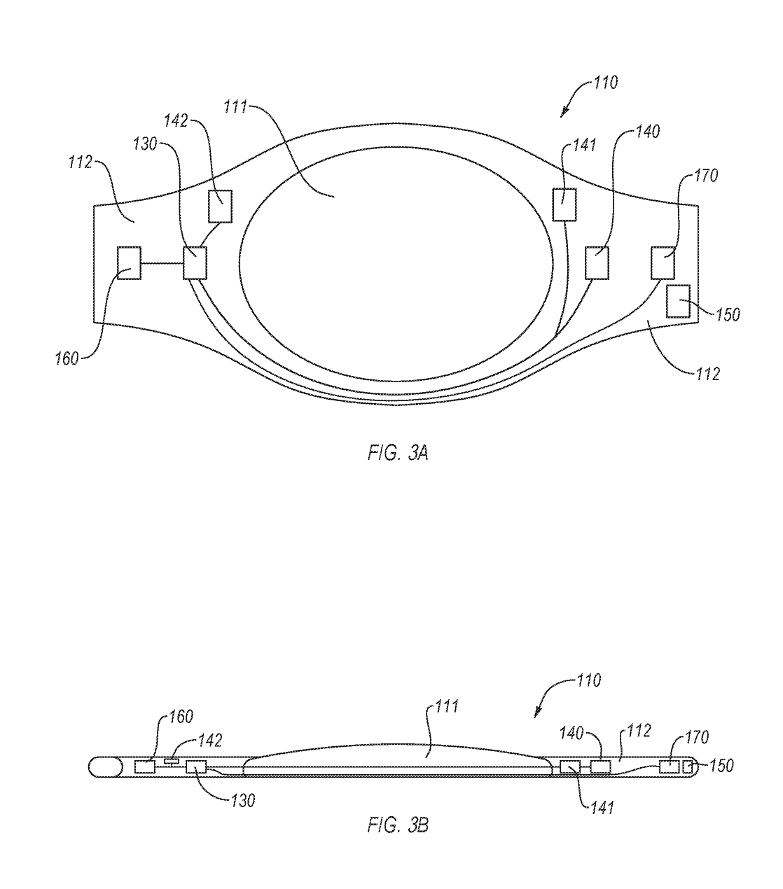

FIGS. 3A and 3B illustrate the switchable lens device 110, according to an embodiment. FIG. 3A is a top view of the switchable lens device 110 and FIG. 3B is a side view of the switchable lens device 110. It should be appreciated, however, that the switchable lens 110′ (FIGS. 1A-1B) can be similar to or the same as the switchable lens device 110. Generally, the switchable lens device 110 can include a switchable lens 111 and haptics 112 connected to or integrated with the switchable lens 111. In an embodiment, the haptics 112 can be positioned on or secured to one or more structures in the eye, thereby positioning or securing the switchable lens device 110 in the eye.

For example, the haptics 112 can be positioned on the ciliary body or muscles or in or on the capsular bag of the natural lens of the eye. The switchable lens 111 can be located laterally in the center of the eye with the haptics 112 extending laterally therefrom. As mentioned above, the switchable lens 111 of the switchable lens device 110 can be switched between two or more optical settings (e.g., two or more focal lengths, to focus light entering the eye from a selected distance on the retina of the eye), thereby augmenting or correcting the vision of the subject.

In an embodiment, the switchable lens device 110 can be substantially fixed within the eye (e.g., switchable lens device can be substantially immobile relative to the optical axis of the eye). As such, for example, movement of the eye 20 can result in a corresponding movement of the switchable lens device 110. In particular, as the eye 20 tilts or pivots in the eye socket, the switchable lens device 110 can correspondingly tilt or pivot together with the eye. Furthermore, one, some, or all of the elements or components of the switchable lens device 110 can have a predetermined orientation relative to the eye or relative to the optical axis thereof, as described below in more detail.

In an embodiment, a controller 130 that can include control electrical circuitry can be operably coupled to the switchable lens 111 and can switch or direct switching of the switchable lens 111 between two or more optical settings (e.g., between two or more focal lengths). In an embodiment, the controller 130 can be positioned on or embedded in one or more portions of the switchable lens device 110. For example, a controller 130 can be mounted on, embedded in, or otherwise mechanically connected to the haptics 112 (e.g., as schematically shown in FIG. 3B). Additionally or alternatively, the controller 130 can be mounted on, embedded in, or otherwise mechanically connected to the switchable lens 111 or any other suitable portion of the switchable lens device 110.

As described above, the switchable lens device 110 can include one or more sensors that can detect or sense inputs, events, or conditions that can be related to or associated with the change of vergence between the eyes of the subject (e.g., sensors that can detect movement or direction of movement of the first or second eye of the subject). In an embodiment, the controller 130 can be operably coupled to and can receive detection outputs from a field sensor 140 that can be operably (e.g., mechanically) coupled to one or more portions of the switchable lens device 110 (e.g., to the haptics 112).

Generally, the field sensor 140 can include a single or multiple sensors that can detect presence or changes in a magnetic field. For example, the field sensor 140 can move together with the subject's eye and can detect a change in an established identifiable field (e.g., a magnetic field that can be established at a suitable location relative to the field sensor 140 or relative to the switchable lens device). Additionally or alternatively, the switchable lens device 110 can include a field source 150 that can establish an identifiable field. Another field sensor (e.g., located externally to the switchable lens device 110) can detect the change in the field as the field source 150 moves together with the switchable lens device 110 that can move together with the eye of the subject. Field sources, field sensors, and switchable device and controller configurations that are suitable for determining the change in vergence between the first and second eyes of the subject (e.g., for switching the switchable lens from the first optical setting to the second optical setting, such as from the first focal length to the second focal length) are disclosed in more detail in U.S. patent application Ser. No. 14/807,719, the entire content of which is incorporated herein by this reference.

In an embodiment, the switchable lens device 110 can include an acceleration sensor 141 that can be operably coupled to the controller 130. Generally, the acceleration sensor 141 can also include a single accelerometer, gyroscope, etc., or multiple accelerometers, gyroscopes, etc., (e.g., two or more of which can be arranged in an array). For example, the acceleration sensor 141 can move together with the switchable lens device 110 (e.g., together with the eye of the subject), and the controller 130 can receive one or more signals from the acceleration sensor 141, which can be related to the movement detected by the acceleration sensors. Moreover, based at least partially on the signals received from the acceleration sensor 141, the controller 130 can determine the change in vergence between the subject's first and second eyes, as described in more detail in U.S. patent application Ser. No. 15/267,526, the entire content of which is incorporated herein by this reference.

In an embodiment, the switchable lens device can include a physiological characteristic sensor (not shown). For example, physiological characteristic sensor can detect one or more physiological characteristics or changes therein, such as glucose concentration (e.g., in the eye of the subject), eye pressure, heart rate, biological proteins present in the eye, or any other biological indicia. In an embodiment, the controller 130 can determine one or more focus-related characteristics based on the activity of the subject's ciliary muscles. For example, the physiological characteristic sensor can detect activity of the ciliary muscle(s) of the subject and can generate one or more signals responsive thereto (e.g., the signal(s) generated by the physiological sensor(s) can be related to the change in vergence between the eyes of the subject). The physiological characteristic sensor can be operably coupled to the controller 130, and the controller 130 can receive one or more signals from the physiological characteristic sensor and can switch or direct switching of the switchable lens 111 based at least partially on one or more signals received from the physiological characteristic sensor.

It should be appreciated that the switchable lens device 110 can include one, some, or all of the sensors described above, which can be operably coupled to the controller 130 and can send one or more signals thereto; the controller 130 can determine or estimate change in vergence between the subject's eyes based at least partially on the signals received from such sensors. Moreover, the switchable lens device 110 can include one or more additional or alternative sensors (e.g., as described below in more detail) that can be operably coupled to the controller 130, and based on signals from which the controller 130 can determine or estimate change in vergence between the subject's eyes. As described above, under some operating conditions or in some instances, the subject may desire to override the optical setting selected by or switched to by the controller 130 or to switch to a different optical setting than the optical setting selected by or switched to by the controller 130 (e.g., responsive to controller 130 making the determination of change in vergence).

For example, the switchable lens device 110 can detect or receive one or more inputs from the subject, which can indicate or can be correlated to a command for switching to a previous optical setting. In an embodiment, as described below in more detail, the switchable lens device 110 (e.g., the controller 130 of the switchable lens device 110) can receive audible or gesture inputs and correlate the inputs to one or more switching or override commands.

In an embodiment, the controller 130 can receive a detection output from an input sensor 142. For example, the detection output can be related or can correspond to one or more commands or selections for one or more optical settings (e.g., for a single optical setting for one or more switchable lenses or for multiple optical settings for two or more switchable lenses). At least partially based on the received detection output, the controller 130 can switch or direct switching of the switchable lens 111 to a selected or predetermined optical setting, such as a previous optical setting.

For example, the controller 130 can correlate the signals received from the input sensor 142 to one or more override commands or optical settings (e.g., default optical settings). As described herein, the input sensor 142 can detect audible input or commands provided by the subject. For example, the subject can provide an audible command that can be detected by the input sensor 142, and, based at least partially on the signals received from the input sensor 142, which can be related to the audible command provided by the subject, the controller 130 can switch or direct switching of the switchable lens 111 to a selected optical setting. Under some operating conditions, the commands provided by the subject can override the optical that was previously determined or selected by the controller 130 (e.g., as mentioned above, the controller 130 can select an optical setting or switch the switchable lens 111 to a selected optical setting based at least partially on one or more non-command inputs, events, or conditions determined by one or more sensors operably coupled to the controller 130).

In an embodiment, the switchable lens device 110 can be configured to switch between two or more discrete optical settings. For example, the switchable lens device 110 can have set or specific focal lengths to which the switchable lens 111 can be switched (e.g., a first focal length for viewing nearby objects and a second focal length for viewing objects at a distance). Similarly, the switchable lens device 110 can be switched between two or more discrete settings for the visible-light transmittance through the switchable lens 111.

It should be appreciated that any optical setting may involve at least one optical parameter, such as focal length, focal or optical power (e.g., magnification), light transmission, focal position, etc. In some embodiments, at least one optical setting includes multiple optical parameters. In an embodiment, the controller 130 can store (directly or indirectly (e.g., the controller 130 can be operably coupled to a storage)) multiple optical settings and corresponding optical parameters thereof. Moreover, the controller 130 can store multiple optical settings and corresponding switching commands (e.g., audible commands or inputs) for switching to each optical setting. For example, the controller 130 can include or can be operably coupled to a table that correlated multiple optical settings with corresponding optical parameters and switching commands (e.g., commands that can be received from the subject) or signals (e.g., signals that can be received from the input sensor 142).

In an embodiment, the controller 130 can switch or direct switching of the switchable lens 111 from a first focal length to a second focal length (e.g., responsive to a command received from the subject or responsive to a non-command input). In an embodiment, at the first focal length, the switchable lens 111 can have a different focal or optical power than at the second focal length. Moreover, the subject can provide input or command (e.g., that can be detected by the input sensor 142), and the controller 130 can change the focal length or the optical power of the switchable lens 111 responsive to the command received from the subject (e.g., overriding previously selected focal length or optical power of the switchable lens 111, as may be selected by the controller 130 responsive to a non-command input).

In an embodiment, the input sensor 142 can detect one or more inputs that can be correlated to a selection of at least one optical setting for the switchable lens 111. For example, as described below in more detail, responsive to the signal(s) received from the input sensor 142, the controller 130 can switch or direct switching of the switchable lens 111 to a selected optical settings (e.g., the controller 130 can generate one or more switching signals that can be based on or responsive to the signal(s) received from the input sensor 142).

In an embodiment, the input sensor 142 can include one or more audio or sounds sensors or detectors (e.g., a microphone). For example, the input sensor 142 can detect an audio input or command, and the controller 130 can receive one or more signals from the input sensor 142, which can correspond to the audio input(s) detected by the input sensor 142. Moreover, the controller 130 can correlate the audio input(s) detected by the input sensor 142 or the signals received from the input sensor 142 to one or more switching commands or to one or more optical settings for the switchable lens 111.

For example, the subject wearing or using the switchable lens device 110 can provide an audible command requesting that the switchable lens device 110 switch the switchable lens 111 from a first optical setting to a second optical setting, or vice versa. The input sensor 142 can detect the audible command provided by the subject, and responsive to the detection of the audible command at the input sensor 142, the controller 130 can receive one or more signals from the input sensor 142. Moreover, as discussed above, the controller 130 can correlate the one or more signals received from the input sensor 142 to one or more optical settings corresponding thereto and requested by the subject. Responsive to the audible command (e.g., from the subject), the controller 130 also can switch the switchable lens 111 from the first optical setting to the second optical setting, from the second optical setting to a third optical setting, and so on, by switching or directing the switching of the switchable lens 111 to the optical setting that corresponds to the command detected by the input sensor 142.

In an embodiment, the controller 130 can correlate an audible command or input (e.g., from the subject) with an optical setting for the switchable lens 111. For example, an audible command can be a voice command. Hence, in an embodiment, the controller 130 can correlate verbal audible commands or sounds, such as subject speaking out loud a phrase, a number, a parameter, etc., with a selected optical setting. In an embodiment, the controller 130 can be programmed or configured to filter out voice commands from persons other than the subject. For example, the controller 130 can be configured or programmed to distinguish the voice of the subject from persons other than the subject.

Additionally or alternatively, audible commands can include non-verbal commands. Generally, the non-verbal commands can include any suitable or repeatable sound or noise that can be associated with a specific optical setting for the switchable lens 111. For example, suitable non-verbal commands can include clacking of teeth, clapping, clicking, etc. The controller 130 can be configured to correlate non-verbal commands to specific or corresponding optical settings. For example, the subject can train the controller 130 to correlate a clicking sound with a first optical setting, and clacking of teeth with a second optical setting.

In an embodiment, a command for switching the switchable lens 111 to a suitable or selected optical setting can involve a series, a sequence, or a pattern of non-verbal commands or sounds. For example, a selected number of repeated claps, clicks, combinations thereof, etc., can be correlated with a selection for a specific optical setting. In an embodiment, the selected number of non-verbal or repeated commands must be provided by the subject within a selected time period. For example, the controller 130 can filter out verbal or non-verbal inputs (or signals received from the input sensor 142, which correspond to the verbal and non-verbal inputs) that do not correspond to a command input (e.g., verbal or non-verbal inputs that do not include or correspond to a selected sound, selected number of repeats of the sound(s) time period within which the sound(s) were repeated, etc.).

For example, the controller 130 can be configured to correlate the input received from the subject or from the input sensor 142 with selected period of time. Moreover, the selected period of time can be the time period from the time of switching of the switchable lens 111 (e.g., to a selected setting based on non-command related signal(s) received from one or more sensors) to the time of receiving suitable input from the input sensor 142 (e.g., the subject's input, such as audible input, or command must be received at the controller 130 within a selected period of time after the controller 130 switched or directed switching of the switchable lens, such as responsive to one or more non-command signals or inputs that can be received from one or more sensors). In other embodiments, the selected period of time can be the time period from the time of switching between an enabled state for sensor-based switching and a disabled state for sensor-based switching, to the time of receiving suitable input from the input sensor 142 (e.g., the subject's input, such as audible input, or command must be received at the controller 130 within a selected period of time after the controller 130 switched or directed switching between an enabled and a disabled state for sensor-based switching.

Hence, for example, the controller 130 can filter out signals received from the input sensor 142, which are received at the controller 130 outside of the selected time period or which do not correspond to a command for switching the switchable lens 111 from one optical setting to another. It should be appreciated that, as described below in more detail, the switchable lens device 110 can include any number of suitable sensors or detectors that can detect any number of inputs (e.g., inputs from the subject using the switchable lens device 110); the sensors or detectors can generate one or more signals responsive to such inputs, and the signals can be correlated by the controller 130 to one or more optical settings.

As mentioned above, the optical settings for the switchable lens 111 of the switchable lens device 110 can involve any number of suitable optical properties. For example, the switchable lens 111 can be switchable between two or more focal lengths. In an embodiment, the switchable lens 111 can be configured to switch between at least two different optical settings (e.g., between two different focal lengths) responsive to one or more electrical inputs. Hence, for example, the controller 130 can switch or direct switching of the switchable lens 111 between two or more optical settings by generating a suitable electrical signal. That is, the switchable lens 111 can be an electrically-modifiable diffractive lens, such as the electrically-modifiable lenses described in more detail in U.S. application Ser. No. 14/807,673, the entire content of which is incorporated herein by this reference. Additionally or alternatively, the switchable lens 111 can be liquid crystal lens (e.g., a liquid crystal lens with an electrically tunable focal length).

In an embodiment, the controller 130 can be configured to compare or correlate the inputs from the subject or the signals received from the input sensor 142 to one or more approved inputs (e.g., the controller 130 can receive or include one or more approved inputs that can be stored in the controller 130, such as in a table or database). For example, the approved inputs can correspond to one or more optical settings (e.g., first, second, etc., optical settings can be correlated to corresponding first, second, etc., approved inputs). In an embodiment, the controller 130 can distinguish inputs from the subject that correspond to approved inputs from other inputs that do not correspond to approved inputs (e.g., to differentiate between a command provided by the subject to switch to an optical setting, such as a previous optical setting, and a non-command sound or gesture made by the subject). Generally, approved inputs may be in any suitable form (e.g., the controller 130 can differentiate a first signal from the input sensor 142, which can be correlated to an approved input, from a second signal from the input sensor 142, which does not correlate to an approved input).

In an embodiment, one or more approved inputs can be based on one or more environmental conditions or settings (e.g., of the subject or of the switchable lens device 110). For example, one or more sensors or detectors of the switchable lens device 110 (e.g., the acceleration sensor 141, physiological characteristics sensor, etc.) can detect one or more environmental conditions. Generally, environmental conditions can include any suitable condition that can facilitate correcting a potentially inaccurate determination of an optical setting by the controller 130. For example, environmental conditions can include a determination of the speed of movement of the subject, which can be correlated to the location of the subject (e.g., standing, walking, running, driving, flying, etc.). For example, the controller 130 can switch or direct switching of the switchable lens 111 responsive to one or more non-command signals from one or more sensors (e.g., responsive to one or more signals received from the field sensor 140), where the second focal length focuses the switchable lens 111 on an object closer that at the first focal length. The subject can provide one or more audible or gesture inputs (as described herein) to switch to the first focal length or to another focal length. For example, an input from the subject can be correlated by the controller 130 to the first focal length and to the second focal length (e.g., due to the noise, such as in an audible input). The controller 130 can correlate the input or distinguish the signal based on one or more environmental conditions (e.g., where the controller 130 determines that the subject is driving, the controller 130 can favor or weigh heavier the probability that the input received from the subject corresponds to the first focal length, focusing on the farther object).

In an embodiment, the subject may request the controller 130 to switch the switchable lens 111 to a first optical setting, under which the switchable lens 111 can have a first position relative to the haptics 112, a first focal length, and a first visible-light-transmittance setting. As mentioned above, any number of suitable commands or inputs (e.g., audible input, gesture inputs (described below), etc.) can be provided to the controller 130 (e.g., via the detection at the input sensor 142, which the subject can input the switching command or request to change from one optical setting to another). In an embodiment, the input(s) or commands received from the subject can include information corresponding to an optical setting for the switchable lens 111 (e.g., the optical setting that was set for the switchable lens 111 before the switchable lens 111 was switched, such as responsive to one or more non-command signals received by the controller 130 from one or more sensors). For example, the subject may speak a phrase (e.g., “first setting”) that can be detected by the input sensor 142, and, responsive to the signals received from the input sensor 142, the controller 130 can generate a switching signal, switch, or direct switching of the switchable lens 111 to the selected or corresponding optical setting.

Additionally or alternatively, the audible input or spoken command can refer or correspond to a previous optical setting (e.g., optical setting that was set for the switchable lens 111 before the change by the controller 130, such as before the change responsive non-command input, event, or condition). For example, the controller 130 can correlate one or more signals received from the input sensor 142 detecting the subject speaking a suitable word or phrase (e.g., “go back”) to one or more previously set optical settings. For example, the controller 130 can store one or more previous optical settings that were set for the switchable lens 111 and can retrieve the store optical setting(s) responsive to the corresponding commands received from the subject or signals received from the input sensor 142. Moreover, in an embodiment, the controller 130 can switch or direct switching the switchable lens 111 to the corresponding selected or retrieved optical setting (e.g., corresponding to the command received from the subject).

As described above and further described below, the switchable lens device 110 can include any number of suitable sensors (e.g., field sensor 140, acceleration sensor 141, video detector, etc.) that can detect any number of non-command inputs, events, or conditions and provide corresponding signals to the controller 130 for determining one or more suitable optical settings for the switchable lens 111. In an embodiment, the controller 130 can receive one or more inputs or commands from the subject (e.g., audible commands as described above) that the controller 130 can correlate to a selection of one or more sensors or inputs, events, or conditions detected thereby that the controller 130 can use or accept for determining an optical setting (e.g., focal length) for the switchable lens 111. For example, responsive to one or more commands or inputs received from the subject, the controller 130 can block or ignore signals from one or more sensors (e.g., the controller 130 can ignore signals received from the acceleration sensor 141 and accept signals received from the field sensor 140 for determining the focal length for the switchable lens 111).

In an embodiment, the controller 130 can be configured or programmed according to a plurality of algorithms that can be selected by the controller 130 for determining a suitable optical setting for the switchable lens 111. Generally, a selected algorithm for determining the suitable optical setting for the switchable lens 111 can involve any number of steps or operations that can be performed by the controller 130, and which can involve or require input or signals from any number of suitable input sources (e.g., sensors) and any number of computational or correlative operations therewith (e.g., mathematical operations, operation correlating the input(s) or signal(s) to one or more stored values, etc.). In an embodiment, the controller 130 can be programmed or configured to determine the suitable optical setting according to any number of selectable algorithms (e.g., that can be selected by the subject). For example, the controller 130 can base the determination of the optical setting at least partially on the environmental conditions detected by one or more sensors. Additionally or alternatively, the controller 130 can receive one or more inputs or commands from the subject, indicating to select a specific algorithm, according to which the controller 130 can determine or switch to an optical setting (e.g., the controller 130 can process one or more inputs from sensor(s) based on the selected algorithm(s)).

In an embodiment, a first algorithm can include a first set of operations that can require a first set of inputs, and a second algorithm can include a second set of operations that can require a second set of inputs. For example, the second set of inputs can be different than the first set of inputs. For example, the first set of inputs can include signals from an acceleration sensor and from a field sensor, and the second set of inputs can include signals only from the field sensor (e.g., the signals from the acceleration sensor may be disregarded when the controller 130 determined the optical setting for the switchable lens 111 based on the second algorithm).

In an embodiment, the subject can provide one or more audible or gesture inputs (e.g., as described herein) that can be detected by the input sensor 142, which can be correlated by the controller 130 to one or more command. For example, based on the one or more commands from the subject, the controller 130 can select a suitable algorithm (e.g., the command can include an algorithm selection or one or more parameters that can be correlated by the controller 130 to a suitable algorithm selection). Likewise, based on the command(s) received from the subject, the controller 130 can switch from basing the determination of the optical setting on one algorithm to another (e.g., switch from using the first algorithm to using the second algorithm or vice versa). Moreover, responsive to the command received from the subject, the controller 130 can switch the optical setting of the switchable lens 111 (as described herein) and switch from one algorithm to another (e.g., based on the same command or input received from the subject).

In an embodiment, the controller 130 can be configured to stop sensor-based switching of the switchable lens 111 (e.g., to stop switching the switchable lens 111 according to one or more algorithms described herein). For example, the controller 130 can be configured to stop sensor-based switching of the switchable lens 111 responsive to one or more inputs or commands received from the subject. Furthermore, the controller 130 can be configured to resume sensor-based switching of the switchable lens 111 (e.g., responsive to one or more additional or alternative inputs or commands received from the subject, responsive to one or more detected conditions or events, etc.). For example, the controller 130 can switch between an enabled state for sensor-based switching and a disabled state for sensor-based switching. Hence, when a state setting is set to an enabled state, the controller 130 can be configured to direct sensor-based switching an optical setting of the at least one switchable lens from the first optical setting to the at least a second optical setting or from the second optical setting to the first optical setting responsive to the one or more sensor signals. Conversely, for example, when the state setting is set to a disabled state, for example, the controller can maintain the optical setting unchanged.

In an embodiment, the switchable lens device 110 can include at least one output device 170. For example, the output device 170 can provide an indication to the subject that the controller 130 received an input from the subject or accepted, or executed a command for switching from one optical setting to another (e.g., a command that corresponds to the input received from the subject). In an embodiment, the controller 130 can be operably coupled to the output device 170 and can send one or more signals thereto, indicating that the controller 130 has received one or more signals from the input sensor 142, which correspond to at least one command for switching the switchable lens 111 from one optical setting to another. For example, responsive to the signal(s) received (directly or indirectly) from the controller 130, the output device 170 can generate an output that is identifiable by the subject.

In an embodiment, the output device 170 can include a speaker that can generate an audible feedback to the subject. For example, the speaker can produce one or more selected non-verbal sounds, such as beeping, clicking, etc., or one or more selected verbal sounds, such as a spoken word or phrase (e.g., “selection accepted”). It should be appreciated, however, that the output device 170 can include any number of output devices suitable for providing feedback to the subject, which is suitably or sufficiently identifiable by the subject.

In an embodiment, the output device 170 can include a haptic feedback generator. For example, the output device 170 can vibrate, pulse, etc., in a manner that can be detected or identified by the subject. Moreover, a selected combination or pattern of haptic feedback can provide the subject with a specific feedback or identification, which can indicate to the subject that the controller 130 received an input from the subject or accepted, or executed a command for switching from one optical setting to another. For example, a short pulse can indicate that the subject's command was accepted by the controller 130, and a longer pulse can indicate that the subject's command was not accepted by the controller 130. Additionally or alternatively, the output device 170 can include a visible indicator. For example, the output device 170 can include a visible indicator. For example, the visible indicator can include lights of one or more selected wavelengths. In an embodiment, the light(s) can be produced by a light generator (e.g., LED) that can generate feedback of suitable color, intensity, pattern (e.g., Morse code), etc., to provide suitable feedback to the subject.

In an embodiment, the switchable lens device 110 can include a communication device 160 (e.g., the controller 130 can be operably coupled to the communication device 160). The communication device 160 can be mounted on or embedded in one or more portions of the switchable lens device 110. For example, the communication device 160 can be embedded in or mechanically connected to the haptics 112 or the switchable lens 111 of the switchable lens device 110.

In an embodiment, the communication device 160 can be wireless (e.g., the communication device 160 can be a transmitter or a transceiver) or wired. For example, a wireless (e.g., RF-based or US-based) connection can be established between the communication device 160 and another or additional communication device. Alternatively, the communication device 160 and another communication device can have a wired connection therebetween. For example, an electrical conductor connecting the communication device 160 and another communication device can be implanted in or near the eyes of the subject. In any embodiment, the communication device 160 can be operably coupled to the additional communication device, such as to send data therebetween.

In an embodiment, the controller 130, input sensor 142, output device 170, or communication device 160 can be operably coupled or connected to a power source. For example, the power source can include a rechargeable energy storage device or battery (not shown) that can be mounted on or embedded in the switchable lens device 110. The battery can be wirelessly recharged (e.g., a wireless or inductive charger can recharge the battery). In an embodiment, the battery can be operably connected to a photovoltaic cell that can be mounted on or embedded in the switchable lens device 110. Alternatively or additionally, the battery can be operably connected or coupled to a charge port that can be configured to accept a charging device. In any event, the power source can power one or more of the controller 130, input sensor 142, output device 170, or communication device 160.

In an embodiment, the power source may include a parasitic power device, such as an induction coil, one or more photocells, thermoelectric device, or any other device configured to harvest energy from a subject or the environment. For example, the induction coil can include a channel having a magnet therein, the channel passing the induction coil upon movement of the subject (e.g., eye-movement or blinking). In an embodiment, an induction coil can be disposed in the eye of a subject (e.g., in or adjacent to the switchable lens) and a corresponding magnet may be positioned on an adjacent part of the subject (e.g., an eyelid or bridge of the nose) whereby movement of the eye or eyelid can cause a current in the induction coil.

As described above, the switchable lens system can include a single switchable lens device or multiple switchable lens devices (e.g., a switchable lens device can be located in one or in both eyes of the subject). Generally, the switchable lens devices of the switchable lens system can be similar to or the same as the switchable lens device 110. It should be appreciated, however, that any of the switchable lens devices included in the switchable lens systems described herein can include or can be operably coupled to any number of controllers, input sensors, output devices, or combinations thereof, which can be similar to or the same as the controller 130, input sensor 142, output device 170, and communication device 160.

While the switchable lens device 110 is described as including the controller 130, input sensor 142, output device 170, and communication device 160, configurations of the switchable lens device 110 can vary from one embodiment to the next. For example, the switchable lens device 110 can include the switchable lens 111, and the controller 130, input sensor 142, or output device 170 can be positioned externally to the switchable lens device 110. For example, the controller 130, input sensor 142, or output device 170 can be included in an external device, such as a personal electronic device (e.g., subject's smart phone). Moreover, as described above, the subject can wear switchable lens devices in both eyes. Hence, for example, a first switchable lens device can include the switchable lens 111, controller 130, input sensor 142, output device 170, communication device 160, or any combination thereof, while the second switchable lens device can include a switchable lens and a communication device operably coupled to the communication device 160 and configured received switching signals therefrom (e.g., the controller 130 can generate switching signals or direct switching of the switchable lens 111 and of a second switchable lens).

In the illustrated embodiment, the switchable lens 111 is an intraocular lens. It should be appreciated, however, that the switchable lens 111 can be any other suitable lens, as described below in more detail. For example, the switchable lens can be included in or can form a part of a contact lens that can be worn by the subject. Additionally or alternatively, the switchable lens can be included in one or more devices positionable externally to the subject's eye. As described below in more detail, for example, the switchable lenses can be included in spectacles, goggles, shields, etc.

Moreover, the input sensor 142 can include any number of sensors suitable for detecting subject's input. For example, the input sensor 142 can include one or more photocells that can detect the subject's use of eye blinking as an input signal. For example, as described below in more detail, the input sensor 142 can include one or more motion sensors (e.g., accelerometers) that can detect movements or gestures of the subject (e.g., movements of subject's head) that can be made by the subject as an input of a command to the controller 130. In an embodiment, the controller 130 can correlate one or more signals received from accelerometer(s) to one or more commands for switching the switchable lens 111 (e.g., as described above in connection with audible commands). For example, a sequence of nods, turns, etc., of subject's head can be detected by the input sensor 142, and responsive to such detection, the controller 130 can switch the switchable lens 111 to a corresponding optical setting.

Moreover, the controller 130 can receive a one or more inputs from one or more sensors configured to detect one or more physiological characteristics sensor, etc. The detection input(s) from the sensor(s) can be related or correspond to the vergence rotation between the subject's eyes. At least partially based on the received output(s), the controller 130 can switch the lens 111 to a suitable or predetermined focal length. For example, the controller 130 can be operably coupled to and can receive input(s) from the physiological characteristic sensor(s) that can be positioned on or embedded in one or more portions of the switchable lens device 110.

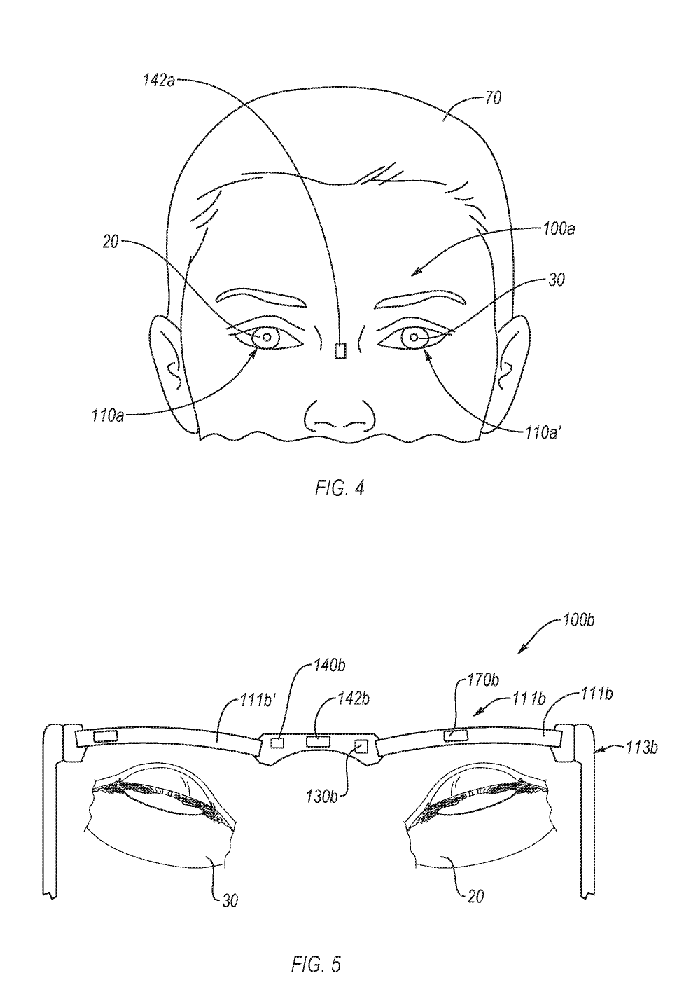

In the illustrated embodiment, controller 130 is mechanically coupled or connected to the switchable lens device 110. Similarly, the input sensor 142 and output device 170 are physically positioned on or included in the switchable lens device 110. Again, however, controllers, input sensors, output devices, or combinations thereof, can be positioned remotely of the switchable lens device, at any number of suitable locations or positions. FIG. 4 illustrates a switchable lens system 100a, according to an embodiment. The switchable lens system 100a can include switchable lens device 110a and switchable lens device 110a′, which can be positioned in the eyes 20 and 30, respectively, of the subject 70. Except as otherwise described herein, the switchable lens device 110a and switchable lens device 110a′ and their respective elements and components can be similar to or the same as any other switchable lens device described herein and their elements and components.

As mentioned above, the switchable lens device 110a or the switchable lens device 110a′ can include respective switchable lenses that can be controlled by a single controller or each of which can be controlled by a separate controller. For example, the switchable lens system 100a can include a controller that can receive signals from any number of suitable sensors, and the received signals can be related to the change in vergence (e.g., as described above), such that the controller can determine one or more optical settings for the switchable lens devices 110a, 110a′ or can switch or direct switching of the switchable lenses (of the respective switchable lens devices 110a, 110a′) responsive to such signals. Furthermore, the switchable lens device 110a or the switchable lens device 110a′ can include fewer elements or components than the switchable lens device 110 (FIGS. 3A-3B).

In an embodiment, the switchable lens system 100a can include an input sensor 142a that can be positioned externally to the switchable lens device 110a and to the switchable lens device 110a′. In the illustrated embodiment, the input sensor 142a is positioned between the eyes 20, 30 of the subject 70 (e.g., the input sensor 142a can be implanted under the skin of the subject 70). It should be appreciated that the input sensor 142a can be positioned at any number of suitable locations. Moreover, the input sensor 142a can be operably coupled to one or more controllers of the switchable lens system 100a; the controller(s) can receive one or more signals from the input sensor 142a (e.g., as described above) and can switch or direct switching of the switchable lenses of the switchable lens device 110a or switchable lens device 110a′. Additionally or alternatively, any other element or component of the switchable lens device 110a or switchable lens device 110a′ can be positioned externally thereto.

As mentioned above, switchable lenses can be included in one or more devices positionable externally to the eyes of the subject. Moreover, any of the elements or components described above in connection with the switchable lens can be included in or mechanically connected to the switchable lens device that is positionable externally to the subject's eyes (e.g., in spectacles). FIG. 5 illustrates a switchable lens system 100b, according to an embodiment. Specifically, the switchable lens system 100b is configured as spectacles. The switchable lens system 100c can include switchable lens device 110b that includes switchable lens 111b and switchable lens 111b′ secured in a frame 113b, with the switchable lens 111b and switchable lens 111b′ positionable in front of respective eyes 20, 30 of the subject. Except as otherwise described herein, the switchable lens device 110b and its elements and components can be similar to or the same as any other switchable lens device described herein and their elements and components.

For example, the switchable lens system 100b can include controller 130b and input sensor 142b that can be similar to or the same as the controller 130 and input sensor 142 of the switchable lens device 110 (FIGS. 3A-3B). Moreover, the switchable lens system 100b can include an output device 170b. For example, the output device 170b can produce audible signals (e.g., selected sounds, speech-like sounds, etc.), visually identifiable signals (e.g., lights, images, etc.), or tactile signals (e.g., vibrations). For example, the output device 170b can be positioned near the eye 20, such that the signals from the output device 170b are visible to the eye 20. In an embodiment, the output device 170c can be positioned on or near the switchable lens 111b (e.g., the light emitted from the output device 170b can be at least partially transmitted through the switchable lens 111b). Hence, the signals from the output device 170b can be received by the subject.

In the illustrated embodiment, the controller 130b and input sensor 142b can be included in or mechanically connected to the frame 113b of the spectacles of the switchable lens system 100b. It should be appreciated, however, that the controller 130b, input sensor 142b, or output device 170b can be positioned on any portion of the switchable lens device 110b (e.g., on any portion of the frame 113b, on the switchable lens 111b, on the switchable lens 111b′, etc.). Additionally or alternatively, the controller 130b, input sensor 142b, or output device 170b can be positioned externally to the switchable lens device 110b (e.g., the controller 130b, input sensor 142b, or output device 170b can be included in or attached to a device that can be configured to be carried by the subject, such as a smart phone).

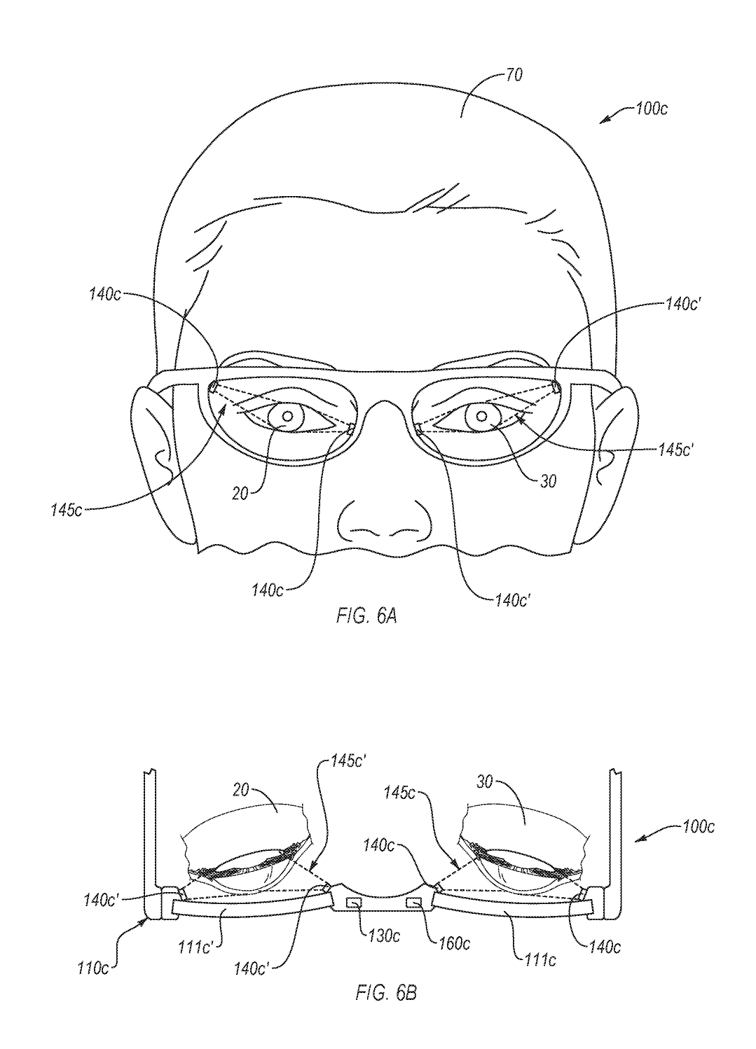

As described above, the switchable lens system or switchable lens device can receive one or more non-audible inputs, such as gestures from the subject and can switch to a selected or predetermined optical setting, such as to the previously set optical setting of the switchable lens 111b or 111b′. For example, the switchable lens system or switchable lens device can include one or more detectors configured to receive or detect gestures provided by the subject. FIGS. 6A-6B illustrate a switchable lens system 100c, according to an embodiment. Except as described there, the switchable lens system 100c and its elements and components can be similar to or the same as any of the switchable lens systems described herein and their corresponding elements and components.

In an embodiment, the switchable lens system 100c can include a switchable lens device 110c that is configured as spectacles. The switchable lens device 110c can include a controller 130c and communication device 160c that can be similar to or the same as the controller 130 and communication device 160 (FIGS. 3A-3B). In an embodiment, the switchable lens system 100c can include one or more sensors 140c or one or more sensors 140c′ that can be input sensors or any number of suitable sensors configured to detect one or more gestures of the subject 70. For example, the input sensors 140c or input sensors 140c′ can include or comprise one or more cameras configured to capture within their respective fields-of-view one or more portions of the body of the subject 70. In an embodiment, the cameras of the sensors 140c can have a field-of-view 145c and the cameras of the sensors 140c′ can have a field-of-view 145c′. In an embodiment, the field-of-view 145c can be suitably sized and configured to capture movement of the eye 20 and the field-of-view 145′ can be suitably sized and configured to capture movement of the eye 30.

In an embodiment, as described above, the controller 130c can be operably coupled to the sensors 140c or to the sensors 140c′ and can receive signals therefrom. For example, signals received at the controller 130c from the input sensors 140c or input sensors 140c′ can be related by the controller 130c to movement of one or more portions the eye 20 or eye 30 of the subject 70. Hence, in an embodiment, the controller 130 can be configured to determine change in vergence between the eyes based at least partially on the detected movement thereof, as described in more detail in U.S. patent application Ser. No. 15/074,606 the entire content of which is incorporated herein by this reference.

In an embodiment, the controller 130c can determine one or more focus-related characteristics based on the change in size of the pupil of the subject's eye. In an embodiment, the switchable lens system 100c or switchable lens device 110c can include one or more sensors (e.g., sensors 140c, 140c′) that can be positioned and configured to determine the size of the pupil(s) of the respective eyes of the subject or change in the size of the pupil(s). For example, the controller 130c can be configured to correlate the change in size of the pupil (e.g., based on the signals received from the sensors 140c, 140c′) to a change in vergence between the eyes of the subject (e.g., as the subject attempts to change the focus of the eyes from a closer object to a farther object or vice versa). As described above, responsive to the determined change in vergence between the eyes, the controller 130c can change or direct changing of one or more optical settings of the switchable lens 111c or switchable lens 111c′ (e.g., change focal length(s) of the switchable lens 111c or switchable lens 111c′).

In an embodiment, the controller 130c can determine one or more focus-related characteristics based at least partially on the light reflection from the retina (or retinae) of the subject's eye(s). Moreover, the controller 130c can determine one or more focus-related characteristics based at least partially on the light characteristics surrounding the subject (e.g., based at least partially on the environment proximate to the subject and the ambient illumination level thereof). For example, the sensors 140c or 140c′ can detect the light reflected from subject's retinae or the level of ambient illumination and can generate one or more signals related thereto, which can be received at the controller 130c. For example, at least partially based on the signals, the controller 130c can determine the change in vergence between the eyes of the subject and can change or direct changing of the corresponding optical settings of the switchable lens 111c or switchable lens 111c′ (e.g., change focal length(s) thereof).

Moreover, in an embodiment, the controller 130c can be configured to distinguish movements of the eye 20 or of the eye 30 that correspond to one or more gesture commands for changing an optical setting of the switchable lens 111c or of the switchable lens 111c′ from movements of the eye 20 or eye 30 that do not correspond to a gesture command for changing an optical setting. As described above, the controller 130c can correlate a gesture command (detected by the sensors 140c or 140c′ and received as one or more signals from the sensors 140c or 140c′ by the controller 130c) to an optical setting selected by the subject 70 for the switchable lens 111c or switchable lens 111c′, such as to switch the switchable lens 111c or switchable lens 111c′ to the previously set optical setting (e.g., to the optical setting selected and set by the controller 130c for the switchable lenses 111c, 111c′ based on the determined or estimated change in vergence between the eyes 20 30 (e.g., from the optical setting that was selected by the controller 130c without a command from the subject 70).

In an embodiment, the fields-of-view 145c or 145c′ can be configured such that the sensors 140c or sensors 140c′, respectively, can capture blinking of the eye 20 or eye 30. As mentioned above, a selected sequence of blinks of the eye 20 or of the eye 30 can correspond to a command for switching an optical setting of the switchable lens 111c or switchable lens 111c′. Hence, for example, the controller 130c can receive signals from the sensors 140c or sensors 140c′ and can correlate the signals received from the sensors 140c or sensors 140c′ to one or more commands for changing to a selected optical setting for the switchable lens 111c or switchable lens 111c′. In an embodiment, as described above, responsive to the signals received from the sensors 140c or input sensors 140c′, the controller 130c can change optical setting of the switchable lens 111c or switchable lens 111c′. Additionally or alternatively, the selected movements of the eye 20 or eye 30 or movement patterns can correspond to a command for changing to a selected optical setting. In any event, for example, responsive tone or more inputs or commands received from the subject, such as audible or gesture inputs or commands, the controller 130c can be configured to switch the switchable lens 111c or switchable lens 111c′ from a first optical setting to another optical setting (e.g., to a previous optical setting or to a different optical setting, generally).

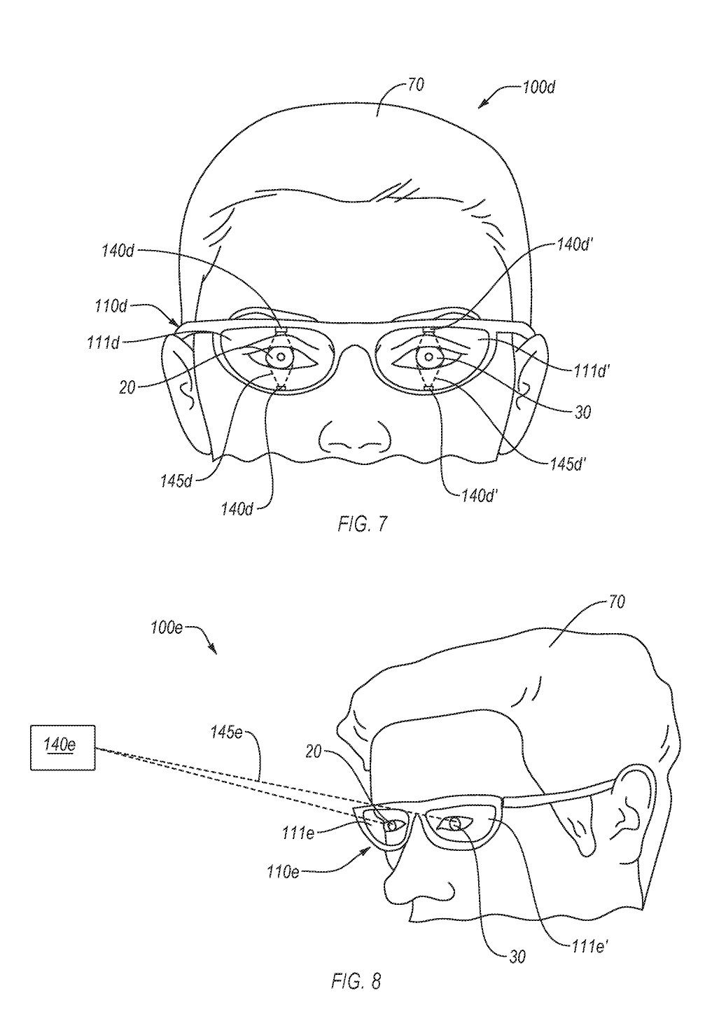

In an embodiment shown in FIGS. 6A-6B, the fields-of-view 145c and 145c′ are oriented generally horizontally relative to the eyes 20, 30. It should be appreciated, however, that positions of the cameras of the sensors 140c, 140c′ and the orientation of the corresponding fields-of-view 145c and 145c′ can vary from one embodiment to the next. FIG. 7 illustrates a switchable lens system 100d, according to an embodiment. Except as otherwise described herein, the switchable lens system 100d and its elements and components can be similar to or the same as any of the switchable lens systems described herein (e.g., similar to or the same as the switchable lens system 100c (FIGS. 7A-7B)).

In an embodiment, the switchable lens system 100d can include switchable lens device 110e that can have sensors 140d and sensors 140d′ (e.g., as described above, the sensors 140d and sensors 140d′ can include one or more cameras). In the illustrated embodiment, fields-of-view 145d and 145d′, of the corresponding sensors 140d, 140d′, can be aligned generally vertically relative to the eye 20 and eye 30, respectively. For example, similar to the fields-of-view 140c, 140c′ (FIGS. 6A-6B), the input sensors 140d or sensors 140d′ can capture movements of one or more portions of the eye 20 or eye 30 (respectively), which can correspond to one or more commands for switching switchable lens 111c or switchable lens 111c′ to a selected optical setting.

As mentioned above, the elements or components of the switchable lens system can be positioned externally to the switchable lens device. FIG. 8 illustrates a switchable lens system 100e, according to an embodiment. Except as described herein, the switchable lens system 100e and its elements and components can be similar to or the same as any of the switchable lens systems described herein. In the illustrated embodiment, the switchable lens system 100e can include switchable lens device 110e that includes switchable lens 111e and switchable lens 111e′, which can be positioned in front of the eye 20 and eye 30, respectively.

In an embodiment, the switchable lens system 100e can include sensor 140e that can be positioned externally to the switchable lens device 110e. For example, the sensor 140e can include at least one camera that can be positionable such that the field-of-view 145e of the camera of the sensor 140e can capture movement of one or more parts of the subject 70. For example, the field-of-view 145e can be suitable to capture movement of the eye 20 or eye 30 of the subject, size of the pupil of the eye 20 or eye 30, light reflected from the retina of the eye 20 or eye 30, level or intensity of ambient light surrounding the subject 70, etc., and can generate one or more signals responsive thereto. as described above. In an embodiment, the controller can receive one or more signals from the sensor 140e and can determine one or more focus-related characteristics based on the received signals.

Moreover, in an embodiment, the field-of-view 145e can be suitable to capture movement of the eyelid of the eye 20 or eye 30 of the subject, movement of the subject's head, hand, or other body parts, etc. As described above, for example, movement of the eye 20 or eye 30 (e.g., movement of the respective eyelids, such as blinking) of the subject 70 can be received as one or more gesture commands or can be correlated to one or more commands for switching the switchable lens 111e or switchable lens 111e′ to a selected optical setting. Moreover, in an embodiment, one or more additional or alternative movements of the subject 70 can be correlated with one or more selected optical settings (e.g., movement of the hand, arm, mouth, etc., can be correlated to a command for a selected optical setting). Hence, for example, the subject 70 can provide any number of suitable inputs to the controller via one or more selected movements, to select a suitable optical setting for the switchable lens 111e or switchable lens 111e′.

In an embodiment, the sensor 140e can include one or more audio sensors (e.g., as described above). Hence, for example, the sensor 140e can receive or detect one or more audio inputs from the subject 70 and can generate one or more signals responsive to the detected audible inputs from the subject 70. For example, as described above, the controller can receive one or more signals from the sensor 140e, which can be related to the audible input provided by the subject 70, and can switch or direct switching of the switchable lens 111e or switchable lens 111e′ at least in part based on the signals received from the sensor 140e.

Generally, the sensor 140e can be positioned at any suitable location (e.g., relative to the subject 70 or relative to the switchable lens device 110e). In an embodiment, the sensor 140e can be positioned on or near a monitor, television, etc., in a manner that can facilitate detection or sensing of the inputs provided by the subject 70. Additionally or alternatively, the sensors 140e can be operably coupled to or integrated with a device that can be carried by the subject 70 (e.g., in a smart phone).

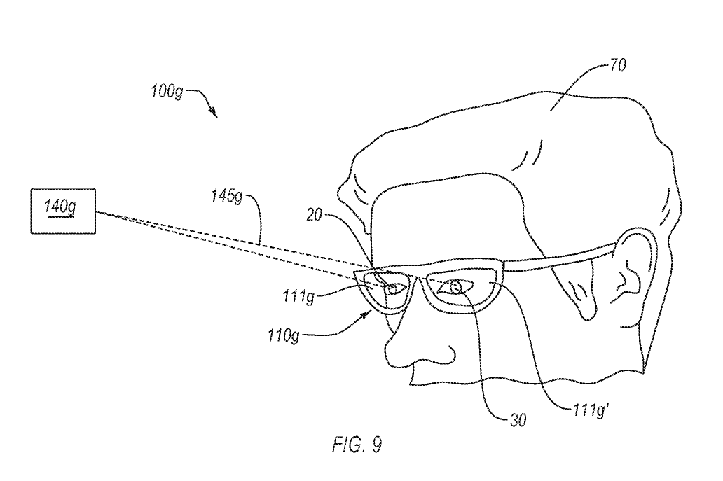

As discussed above, the switchable lens can be included in any number of suitable optical devices that can be used or worn by the subject. FIG. 9 is a schematic side view of a switchable lens system 100g that includes switchable lens devices 110g, 110g′ configured as contact lenses and positioned on the respective eyes 20 and 30, according to an embodiment. Except as otherwise described herein, the switchable lens system 100g and its elements and components can be similar to or the same as any of the switchable lens systems described herein. For example, the switchable lens devices 110g, 110g′ of the switchable lens system 100g can include corresponding switchable lenses 111g and 111g′.

In an embodiment, the switchable lens device 110g can include a controller 130g, a sensor 140g, and a communication device 160g. For example, the sensor 140g can be operably coupled to the controller 130g (e.g., as described above) and can receive or detect one or more inputs from the subject. In an embodiment, the sensor 140g can include a microphone, one or more acceleration or motion detectors (e.g., accelerometers), an optical detector, combinations thereof, etc. For example, the sensor 140g can detect one or more inputs, events, or conditions related to a suitable change to the optical setting of the switchable lens 111g or switchable lens 111g′ (e.g., related to change in vergence between the eyes 20, 30, as described above). Moreover, the sensor 140g can be configured to detect one or more audible or gesture inputs or commands from the subject.

In an embodiment, the communication device 160g can send one or more signals to the switchable lens device 110g′ (e.g., one or more switching signals). The communication device 160g can be operably coupled to another controller or to an input/output interface of a controller (e.g., to a personal electronic device) and can receive signals therefor or send signals thereto. For example, the communication device 160g can receive one or more inputs from an external controller (e.g., from a personal electronic device on the subject) and, responsive thereto, can send one or more signals to the controller 130g. For example, personal electronic device of the subject can detect one or more gesture or audible inputs or commands provided by the subject and can send one or more signals to the communication device 160g responsive thereto. In an embodiment, as described above, the controller 130g can receive one or more signals from the communication device 160g (e.g., corresponding or responsive to the signals received from the external controller, such as personal electronic device) and can generate one or more switching signals from the switchable lens 111g or 111g′.

As described above, the subject can provide one or more gesture or audible inputs that the controller can correlate to or interpret as a command for changing optical setting of the switchable lenses (e.g., for switching to a previous or another optical setting). It should be appreciated, however, that the switchable lens system or switchable lens device can be configured to receive any number of suitable inputs from the subject. For example, the switchable lens system or switchable lens device can include or can be operably coupled to any number of suitable input devices, such as touch input devices (e.g., touch screen, push-button input devices, such as keyboards, etc.), which can receive input from the subject. For example, the controller of the switchable lens device can include or can be operably coupled to a push-button or a keyed input device and can be configured to switch or direct switching of the optical setting of the switchable lens responsive to input received from the input device (e.g., to a previous or another optical setting, as described above).

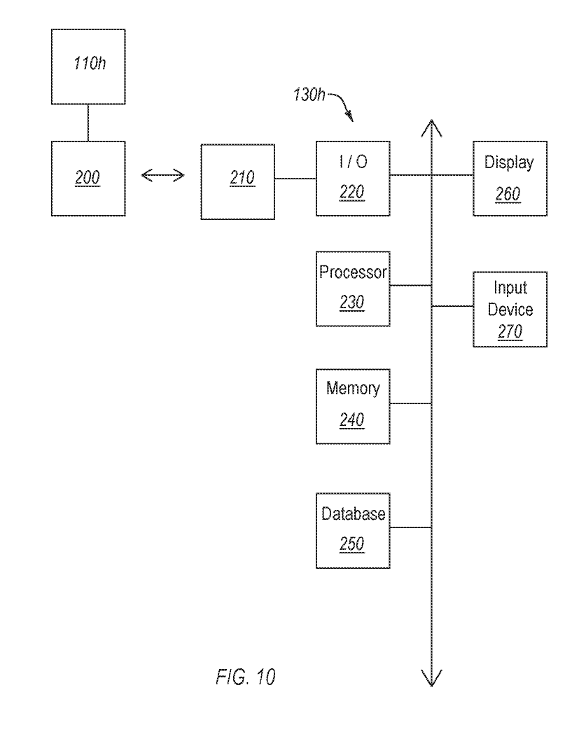

FIG. 10 is a schematic diagram of a switchable lens system 100j that includes a switchable lens device 110j, according to an embodiment. In an embodiment, as shown in FIG. 10, the switchable lens device 110j can include or can be coupled to a communication device 200 (e.g., at least one receiver, transmitter, transceiver, or combinations thereof) that can receive data or instructions related to modification of the focal length(s) of one or more switchable lenses of the switchable lens device 110j. As described above, a switchable lens system can include the controller 130j (e.g., the controller 130j can receive one or more signals and can determine vergence rotation of the eye and/or suitable focal length for the switchable lenses of the switchable lens device 110j). In an embodiment, the controller 130j can include I/O interface 220, processor 230, and memory 240 operably coupled together. In an embodiment, the controller 130j can include a database 250 (e.g., the database 340 and can have data stored in a storage memory of the controller 130j). For example, the controller 130j can store one or more parameters in the database 340 (e.g., the controller 130j can store training or tuning data in the database 340).

In an embodiment, a communication device 210 (e.g., at least one receiver, transmitter, transceiver, or combinations thereof) can be operably coupled to the controller 130j and/or integrated therewith. For example, the communication device 210 can be operably coupled to the communication device 200 (e.g., via wired or wireless connection), such that the switchable lens device 110j and the controller 130e can transmit and receive data from one another. In an embodiment, display 260 and/or input device 270 (e.g., physical or virtual keyboard, microphone, etc.) can be operably coupled to the controller 130j and/or integrated therewith. For example, a user (e.g., a subject using and/or wearing the switchable lens device 110j) can enter input and/or data into the controller 130j, as described herein. Moreover, it should be appreciated that the controller 130j can be operably coupled to and/or incorporated with any number of suitable devices, such as personal electronic devices (e.g., personal computers, smart phones, tablets, etc.) and/or any other computing and/or input devices. It should be appreciated that any of the systems described herein (e.g., multi-focus lens systems, IOL systems, etc.) can have a similar or the same configuration as the system described above and illustrated in FIG. 10.

It will be understood that a wide range of hardware, software, firmware, or virtually any combination thereof can be used in the controllers described herein. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more programs running on one or more processors (e.g., as one or more programs running on one or more microprocessors), as firmware, or as virtually any combination thereof. In addition, the reader will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution.

In a general sense, the various embodiments described herein can be implemented, individually and/or collectively, by various types of electro-mechanical systems having a wide range of electrical components such as hardware, software, firmware, or virtually any combination thereof, and a wide range of components that can impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs.

In a general sense, the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). The subject matter described herein can be implemented in an analog or digital fashion or some combination thereof.

The herein described components (e.g., steps), devices, and objects and the discussion accompanying them are used as examples for the sake of conceptual clarity. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, the reader can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

In some instances, one or more components can be referred to herein as “configured to.” The reader will recognize that “configured to” or “adapted to” are synonymous and can generally encompass active-state components and/or inactive-state components and/or standby-state components, unless context requires otherwise.

While particular aspects of the present subject matter described herein have been shown and described, it will be apparent that, based upon the teachings herein, changes and modifications can be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense the convention (e.g., “a system having at least one of A, B, or C” would include but not be limited to systems that have A alone, B alone, C alone, A and B together, A and C together, B and C together, and/or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, any recited operations therein can generally be performed in any order. Examples of such alternate orderings can include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

While various aspects and embodiments have been disclosed herein, the various aspects and embodiments disclosed herein are for purposes of illustration and are not intended to be limiting, with the true scope and spirit being indicated by the following claims.

What is claimed is:

1. A lens system, comprising:
at least one switchable lens device including at least one switchable lens configured to selectively switch between a first optical setting and at least a second optical setting;
one or more sensors configured to detect at least one focus-related characteristic of one or more of a first eye or a second eye of the subject or at least one focus-related characteristic of an environment proximate to the subject;
a controller operably coupled to the one or more sensors and operably coupled to the at least one switchable lens device, the controller including control electrical circuitry configured to:
receive one or more sensor signals from the one or more sensors;
direct sensor-based switching of the at least one switchable lens from the first optical setting to the at least a second optical setting responsive to the one or more received sensor signals;
receive one or more inputs from the subject; and
after directing sensor-based switching of the at least one switchable lens from the first optical setting to the second optical setting, direct override switching of the at least one switchable lens from the at least a second optical setting to the first optical setting at least partially responsive to the one or more inputs received from the subject.

2. The lens system of claim 1, wherein the control electrical circuitry is configured to direct override switching of the at least one switchable lens from the at least a second optical setting to the first optical setting based on comparison of at least one of the one or more inputs to one or more approved inputs.

3. The lens system of claim 2, wherein the one or more approved inputs correspond to at least one of the first optical setting or the second optical setting.

4. The lens system of claim 2, wherein the one or more approved inputs are based on a type of signal received by the controller and used to direct sensor-based switching.

5. The lens system of claim 2, wherein the one or more approved inputs are based on at least one environmental condition.

6. The lens system of claim 1, wherein the first optical setting includes a first focal length, and the at least a second optical setting includes a second focal length.

7. The lens system of claim 1, wherein the control electrical circuitry is operably coupled to an input device configured to receive the one or more inputs from the subject.

8. The lens system of claim 7, wherein the input device includes one or more of a button, a keyboard, a keypad, a microphone, a motion sensor, a camera, a photodetector, or a touch screen.

9. The lens system of claim 1, wherein the control electrical circuitry is configured to direct switching of the at least one switchable lens from the at least a second optical setting if the one or more inputs are received from the subject within a selected time period after the controller directed switching of the at least one switchable lens from the first optical setting to the at least a second optical setting responsive to the one or more sensor signals.

10. The lens system of claim 9, wherein the control electrical circuitry is operably coupled to an input device and configured to correlate the received input to the time period.

11. The lens system of claim 1, wherein:
the control electrical circuitry is configured to direct switching of the at least one switchable lens from the at least a second optical setting if the one or more inputs are received from the subject within one or more selected time periods after the controller directed switching of the at least one switchable lens from the first optical setting to the at least a second optical setting responsive to the one or more sensor signals; and
each of the one or more selected time periods corresponds to one or more setting conditions.

12. The lens system of claim 11, wherein the one or more setting conditions include at least one of an audible input received from the subject or motion input received from the subject.

13. The lens system of claim 1, wherein the at least one switchable lens device is sized and configured to be placed in at least one of the first eye or the second eye of the subject.

14. The lens system of claim 13, wherein the at least one switchable lens device includes an intraocular lens.

15. The lens system of claim 1, wherein the one or more inputs received from the subject include information corresponding to the first optical setting.

16. The lens system of claim 15, wherein the one or more inputs received from the subject include information corresponding to a focal length of the first optical setting.

17. The lens system of claim 15, wherein the one or more inputs received from the subject include information corresponding to an optical setting set for the at least one switchable lens before the at least a second optical setting.

18. The lens system of claim 1, further including an input device configured to detect one or more of an audio signal or motion of one or more body portions of the subject, and wherein the one or more inputs received from the subject include at least one of an audible input or a motion-based input.

19. The lens system of claim 18, wherein the audible input includes one or more of a verbal voice command, a non-verbal voice command, or a non-vocal audible command.

20. The lens system of claim 18, wherein the motion-based input includes one or more eye motion, eyelid closures, eyelid openings, or eye blinks.

21. The lens system of claim 1, further including an input device configured to receive a keyed input, and wherein the one or more inputs received from the subject include one or more presses of one or more keys to enter the keyed input.

22. The lens system of claim 1, wherein the at least one switchable lens device includes a contact lens.

23. The lens system of claim 1, wherein the at least one switchable lens device include spectacles sized and configured to be worn by the subject.

24. The lens system of claim 1, wherein the controller is mechanically coupled to the at least one switchable lens device.

25. The lens system of claim 1, wherein the controller is included in a personal electronic device.

26. The lens system of claim 1, wherein the one or more sensors include one or more of one or more accelerometers or one or more physiological sensors.

27. The lens system of claim 26, wherein the control electrical circuitry of the controller is configured to generate one or more switching signals to direct switching of the at least one switchable lens from the first focal length to the second focal length at least partially responsive to the one or more signals received from at least one of the one or more accelerometers or the one or more physiological sensors.

28. The lens system of claim 1, further including an energy field source, and wherein the one or more sensors include one or more energy-field sensors.

29. The lens system of claim 28, wherein the energy field source is operably coupled to the first eye or to the second eye of the subject and configured to generate an identifiable energy field, and the one or more energy-field sensors are configured to detect a change in the identifiable energy field responsive to a corresponding change in vergence between the first eye and the second eye of the subject.

30. The lens system of claim 29, wherein the control electrical circuitry of the controller is configured to generate one or more switching signals to direct switching of the at least one switchable lens from the first focal length to the second focal length at least partially responsive to the one or more signals received from the one or more energy-field sensors.

31. The lens system of claim 1, wherein the focus-related characteristic of an environment proximate to the subject includes a range to an object being viewed by the subject.

32. The lens system of claim 1, wherein the focus-related characteristic of one or more of the first eye or the second eye of the subject includes ciliary muscle activity.

33. The lens system of claim 1, wherein the focus-related characteristic of one or more of the first eye or the second eye of the subject includes a change in a size of a pupil.

34. The lens system of claim 1, wherein the focus-related characteristic of an eye of the subject includes differential rotation between the first eye and the second eye of the subject.

35. The lens system of claim 1, wherein the focus-related characteristic of an eye of the subject comprises light reflection from the retina.

36. The lens system of claim 1, wherein the focus-related characteristic of an environment proximate to the subject comprises an ambient illumination level.

37. The lens system of claim 1, wherein the control electrical circuitry of the controller is configured to switch to the first optical setting from another optical setting responsive to one or more inputs received from the subject.

38. The lens system of claim 1, wherein the control electrical circuitry of the controller is configured to switch to the first optical setting from another optical setting responsive to one or more signals received from the one or more sensors.

39. The lens system of claim 1, wherein the control electrical circuitry of the controller is configured to switch to the first optical setting from another optical setting responsive to one or more signals received from an external device.

40. A lens system, comprising:
at least one switchable lens device including at least one switchable lens configured to selectively switch between a first optical setting and at least a second optical setting;
one or more sensors configured to detect at least one focus-related characteristic of one or more of the first eye or the second eye of the subject or of an environment proximate to the subject;
a controller operably coupled to the one or more sensors and operably coupled to the at least one switchable lens device, the controller including control electrical circuitry configured to:
receive one or more sensor signals from the one or more sensors;
direct sensor-based switching an optical setting of the at least one switchable lens from the first optical setting to the at least a second optical setting or from the second optical setting to the first optical setting responsive to the one or more sensor signals, when a state setting is set to an enabled state, and to maintain the optical setting unchanged, when the state setting is set to a disabled state;
receive one or more inputs from the subject; and
after sensor-based switching the optical setting of the at least one switchable lens, at least partially responsive to the one or more inputs received from the subject, change the state setting between a disabled state and an enabled state.

41. The lens system of claim 40, wherein the control electrical circuitry is configured to change from the disabled state to the enabled state if at least one input of the one or more inputs received from the subject is received at the controller within a selected time period after changing the state setting to the disabled state.

42. The lens system of claim 41, wherein the control electrical circuitry is operably coupled to an input device and configured to correlate received input to the time period.

43. The lens system of claim 40, wherein the control electrical circuitry is configured to change from the enabled state to the disabled state when at least one input of the one or more inputs received from the subject is received at the controller within a selected time period after changing the state setting to the enabled state.

44. The lens system of claim 43, wherein the control electrical circuitry is operably coupled to an input device and configured to correlate input received from the input device to the time period.

45. The lens system of claim 40, wherein the control electrical circuitry is configured to change from the enabled state to the disabled state if at least one input of the one or more inputs received from the subject is received at the controller within a selected time period after changing the optical setting between the first optical setting and the second optical setting.

46. The lens system of claim 45, wherein the control electrical circuitry is operably coupled to an input device and configured to correlate input received from the input device to the time period.

47. The lens system of claim 40, further including an input device configured to detect one or more of an audio signal or a motion of one or more body portions of the subject, and wherein the one or more inputs received from the subject include at least one of an audible input or a motion-based input.

48. The lens system of claim 47, wherein the audible input includes one or more of a verbal voice command, a non-verbal voice command, or a non-vocal audible command.

49. The lens system of claim 48, wherein the motion-based input includes one or more eye motion, eyelid closures, eyelid openings, or eye blinks.

50. The lens system of claim 40, further including an input device configured to receive a keyed input, and wherein the one or more inputs received from the subject include one or more presses of one or more keys to enter the keyed input.

51. The lens system of claim 40, wherein the control electrical circuitry of the controller is configured to switch to the first optical setting from another optical setting responsive to one or more inputs received from the subject.

52. The lens system of claim 40, wherein the control electrical circuitry of the controller is configured to switch to the first optical setting from another optical setting responsive to one or more signals received from the one or more sensors.

53. The lens system of claim 40, wherein the control electrical circuitry of the controller is configured to switch to the first optical setting from another optical setting responsive to one or more signals received from an external device.

54-71. (canceled)

72. A lens system, comprising:
at least one switchable lens device including at least one switchable lens configured to selectively switch between a first optical setting and at least a second optical setting;
one or more sensors configured to detect at least one focus-related characteristic of one or more of a first eye or a second eye of the subject or at least one focus-related characteristic of an environment proximate to the subject;
a controller operably coupled to the one or more sensors and operably coupled to the at least one switchable lens device, the controller including control electrical circuitry configured to:
receive one or more sensor signals from the one or more sensors;
direct sensor-based switching of the at least one switchable lens from the first optical setting to the at least a second optical setting responsive to the one or more received sensor signals and based on a first algorithm;
receive one or more inputs from the subject; and
after receiving the one or more inputs from the subject,
stop directing sensor-based switching; or
direct sensor-based switching of the at least one switchable lens from the first optical setting to the at least a second optical setting responsive to the one or more received sensor signals and based on a second algorithm that is different from the first algorithm.

73-76. (canceled)
